(19) United States
(12) Patent Application Publication
(10) Pub. No.: US 2025/0230389 A1
(43) Pub. Date: Jul. 17, 2025

(54) Device and Method for Improving Methane Production in Anaerobic Digestion Through Magnetic Field of Current-Carrying Coil

(71) Applicant: SHANGHAI JIAO TONG UNIVERSITY, Shanghai (CN)

(72) Inventors: Le Zhang, Shanghai (CN); Ronghou Liu, Shanghai (CN)

(21) Appl. No.: 18/762,758

(22) Filed: Jul. 3, 2024

(30) Foreign Application Priority Data
Jan. 12, 2024 (CN) 202410051074.1

(51) Int. Cl. C12M 1/107; C12M 1/00; C12M 1/06; C12M 1/12; C12M 1/42

(52) U.S. Cl. CPC C12M 21/04; C12M 23/06; C12M 23/14; C12M 27/02; C12M 35/06

(57) ABSTRACT

A device for improving methane production in anaerobic digestion through a magnetic field of a current-carrying coil is provided, which relates to the field of microbial anaerobic digestion. The device includes a feed system, a digester, a stirring system, a magnetic field generation system, a magnetic field measurement system, and a gas flow measurement system. A method for improving methane production in anaerobic digestion through a magnetic field of a current-carrying coil is further provided, including the steps of collecting and pretreating digestion feedstock, collecting and pretreating anaerobic digestion inoculum, manufacturing a current-carrying coil-assisted anaerobic digestion system, starting an anaerobic digestion experiment, and confirming experiment results. A magnetic field is generated by evenly winding a current-carrying coil around a digester, which can provide faster electron transfer for methane anabolic reaction, promote methane anabolic process, and improve the methane yield and efficiency.

### CROSS-REFERENCE TO RELATED APPLICATION

This patent application claims the benefit and priority of Chinese Patent Application No. 202410051074.1 filed with the China National Intellectual Property Administration on Jan. 12, 2024, the disclosure of which is incorporated by reference herein in its entirety as part of the present application.

### TECHNICAL FIELD

The present disclosure relates to the field of microbial anaerobic digestion, and in particular to a device and method for improving methane production in anaerobic digestion through a magnetic field of a current-carrying coil.

### BACKGROUND

In recent years, the disposal of a variety of large amounts of organic wastes (such as kitchen waste, agricultural straw, livestock manure, dry branches, and fallen leaves) has attracted great attention from governments in China and other countries. If not treated in a timely and effective manner, the organic wastes can be decomposed in natural environment to produce a large number of pollutants that pollute soil, air, and water, seriously affecting human health.

Anaerobic digestion is a method capable of achieving reduction treatment of organic wastes and energy recovery at the same time, and thus is widely used in organic waste recycling industry. Taking livestock waste as an example, livestock manure enters the anaerobic digestion treatment system as digestion feedstock. Under the synergistic effect of the microbial communities for hydrolysis of microorganisms, acidification of microorganisms, acetoxylation of microorganisms, and methane production by microorganisms, the organic components in the feedstock are gradually transformed into methane-rich biogas, and meanwhile, solid-liquid mixed residues called digestate are produced. Because the digestate still contains nutrients that have not been utilized by digestion microbial communities, the digestates are always made into sterile organic fertilizer for agricultural production. However, livestock manure is a type of organic waste with high nitrogen content, the nitrogen elements often exist in the waste in the form of proteins. During the process of anaerobic digestion, the proteins are gradually decomposed to produce small molecular amino acids, which are further decomposed by the microbial communities to produce ammonium nitrogen (NH₄⁺—N) and nitrate nitrogen (NO₃⁻—N), as well as some free ammonia (NH₃). Excessive ammonia concentration can cause toxic effect on the anaerobic microbial communities, thus inhibiting the normal operation of anaerobic digestion process, and may also lead to the failure of the anaerobic digestion process in serious cases. As the kitchen waste is easy to be decomposed, the accumulation of organic acids produced by decomposition during anaerobic digestion often leads to the decrease in pH value, affecting the normal growth and metabolic function of microbial communities, and inhibiting the process of methane production. For lignocellulosic biomass waste (such as agricultural straw and dry branches and fallen leaves), the complex three-dimensional structure of cellulose, hemicellulose, and lignin makes it difficult for microbial enzymes to function effectively, resulting in low methane synthesis efficiency.

The methane anabolic process involves a series of biochemical reactions, including hydrolysis, acidification, acetoxylation and methanation, and the overall rate of this series of biochemical reactions greatly depends on the transfer rate of electrons between different intermediate metabolites. The main problem with the current methane production technology in anaerobic digestion of organic wastes is that the electron transfer rate is limited in a certain range during the conventional anaerobic digestion process, which cannot meet the transfer rate of electrons required for the synthesis rate of high concentration methane, resulting in low methane production efficiency. The industrial application and popularization of the anaerobic digestion technology in ensuring the bio-natural gas industry in China are seriously restricted. Therefore, there is an urgent need to find a technology capable of effectively promoting the efficiency of methane production in anaerobic digestion of organic wastes.

In addition, the problems existing in the prior art for promoting anaerobic digestion by exciting a magnetic field with an external solid magnet include: (1) In some cases, the solid magnet is placed at the bottom of the digester, such a design is not applicable to all large and medium-sized anaerobic digesters that have already been built. (2) In some cases, the solid magnet is placed next to the digester, but there is a problem is that the excited magnetic field is unevenly distributed, and it is impossible to fully and evenly stimulate the microbial communities in the digester. (3) The problem with the solid magnet used in the prior art is that the intensity of the magnetic field cannot be dynamically adjusted according to actual needs.

Therefore, those skilled in the art are committed to developing a device and method for improving methane production by anaerobic digestion through evenly winding a current-carrying coil outside the anaerobic digester to excite a magnetic field. By evenly winding the current-carrying coil outside the digester, the magnetic field is generated to provide faster electron transfer for methane anabolic reaction. The intensity of the magnetic field can be adjusted by adjusting the current in the current-carrying coil, and thus the best intensity of the magnetic field can be optimized and selected to efficiently and stably promote methane anabolic process.

### SUMMARY

In view of the defects in the prior art, the technical problems to be solved by the present disclosure are that the efficiency of methane production in anaerobic digestion of organic wastes is low, and a magnetic field excited by an external solid magnet cannot be flexibly and dynamically adjusted.

In order to achieve the purpose above, a device for improving methane production in anaerobic digestion through a magnetic field of a current-carrying coil is provided, the device includes a feed system, a digester, a stirring system, a magnetic field generation system, a magnetic field measurement system, and a gas flow measurement system. The feed system is connected to the digester. The stirring system includes stirring paddles and a stirring motor, and the stirring paddles are immersed in digestion feedstock in the digester. The magnetic field generation system is evenly wound around the digester. The magnetic field measurement system is located outside the digester. The gas flow measurement system is connected to an upper portion of the digester.

Further, the feed system includes a feed pool, a feed pump, a connecting pipeline, a feed valve, and an inoculum reservoir. The digestion feedstock is placed in the feed pool, the digestion feedstock is injected into the digester through the feed pump, and anaerobic digestion inoculum is added into the digester from the inoculum reservoir.

Further, the magnetic field generation system includes a constant-current power supply, a power output line, the current-carrying coil, and a power input line. The current-carrying coil is evenly wound around the digester to excite the magnetic field to act on digestion microbial communities inside the digester.

Further, the magnetic field measurement system includes a magnetic field measurement electrode, and a magnetic field measurement instrument host. The magnetic field measurement electrode is placed in the magnetic field of the digester, and configured for measuring actual intensity of the magnetic field and feedback data to the magnetic generation system to control intensity of the magnetic field in the digester. The gas flow measurement system includes a gas flowmeter, and a gas bag, and is configured for evaluating methane yield by anaerobic digestion.

A method for improving methane production in anaerobic digestion through a magnetic field of a current-carrying coil is further provided, the above device is used in the method, and the method includes the following steps:
Step 1, collecting and pretreating the digestion feedstock;
Step 2, collecting and pretreating the anaerobic digestion inoculum;
Step 3, evenly winding the current-carrying coil around the digester;
Step 4, adding the digestion feedstock obtained in Step 1 and the anaerobic digestion inoculum obtained in Step 2 into the digester, turning on the magnetic field generation system and the stirring system, and measuring intensity of the magnetic field by the magnetic field measurement system, adjusting a current in the current-carrying coil to adjust the intensity of the magnetic field generated by the magnetic field generation system to an appropriate level, and starting the anaerobic digestion;
Step 5, during digestion, taking samples to measure digestion efficiency; and
Step 6, after the anaerobic digestion is finished, collecting gas in the gas bag, and measuring methane concentration and carbon dioxide concentration to evaluate average methane yield.

Cleaning the digester after above steps are finished, and repeating Steps 1 to 6 for the next fermentation period.

Further, the digestion feedstock in Step 1 is one of kitchen waste mixture, agricultural organic waste, or livestock manure. A method for pretreating in Step 1 includes one or more steps of screening, drying, high-speed stirring, or high-speed crushing the digestion feedstock.

Further, Step 2 specifically includes the following steps:
Step 2.1, taking out digestion solution from a digester of the same digestion feedstock domesticated in advance as the anaerobic digestion inoculum, and dispensing the anaerobic digestion inoculum into an inoculum reservoir; and
Step 2.2, prior to a formal experiment, starving the anaerobic digestion inoculum for 7 days in advance to release undecomposed organic matters.

An obtained bacterial solution is used as the anaerobic digestion inoculum.

Further, frequency of the magnetic field generation system applied in Step 4 is once a day in an entire digestion process. Treatment time of the magnetic field generation system is 5-15 min each time, an output current of the constant-current power supply of the magnetic field generation system is 1-5.5 A, and an output voltage is 1-30 V. The intensity of the magnetic field is adjusted to 0.3-81.6 mT, digestion temperature is set to 37° C., and rotating speed of the stirring system is set to 500-550 rpm.

Further, a method for taking samples in Step 5 is to take samples every other day, and after taking samples is finished, quantitative feedstock and constant-temperature water at 37° C. are added to ensure that working volume of the digester is the same as the initial one.

Further, the average methane yield is increased by 29.5-35% compared with that without assistance of the magnetic field of the current-carrying coil.

In a preferred embodiment of the present disclosure, the magnetic field is generated by evenly winding the current-carrying coil around the digester, so the electrons in the cells of anaerobic digestion microbial communities become more active under the magnetic field force applied by this external magnetic field, which is more conducive to the gain and loss of electrons and can accelerate the transfer rate of the electrons between different intermediate metabolites, thereby providing faster electron transfer for methane anabolic reaction and achieving a more efficient methane anabolic process.

In addition, a method for improving methane production in anaerobic digestion of organic wastes through forming a magnetic field of a current-carrying coil outside a digester is provided by the present disclosure, which can perfectly solve numerous problems in the prior art for promoting anaerobic digestion through exciting a magnetic field by an external solid magnet. As the current-carrying coil is arranged outside the digester instead of installing the solid magnet at the bottom or side of the digester, the technology provided by the present disclosure can be applicable to all large, medium and small anaerobic digesters that have not been built, or have been built.

Furthermore, a method for dynamically adjusting the magnetic field of the current-carrying coil includes the step of dynamically adjusting the intensity of the magnetic field of the current-carrying coil by adjusting the current in the current-carrying coil according to actual needs for the intensity of the magnetic field. The intensity of the excited magnetic field can be quantitatively regulated by adjusting the current in the current-carrying coil, and the wicked problem that the magnetic field of the solid magnet cannot be dynamically adjusted according to the actual needs for the magnetic field is solved.

Compared with the prior art, the present disclosure has the following beneficial effects:

(1) The problem of promoting methane anabolic rate in anaerobic digestion without introducing any foreign substances into the digester is solved. The problem of low methane synthesis efficiency is innovatively solved, the average methane yield is increased by 29.5-35.0%, and the economy of methane production technology by anaerobic digestion of organic wastes is significantly improved.

(2) The method provided by the present disclosure has good compatibility with different organic wastes (kitchen waste, agricultural straw, and livestock waste), can be easily used on all anaerobic digestion equipment which are not built, under construction, and have already been built, and thus has a good application prospect in the biogas industry.

(3) The device and method provided by the present disclosure are helpful to improve the conversion rate of the feedstock of organic wastes, can promote more efficient utilization of resources of organic wastes, promote the utilization of carbon dioxide in biogas (methane concentration in biogas is increased by 17.9-22.4%, and carbon dioxide concentration in biogas is decreased by 14.7-20.6%), reduce carbon emissions, and help to turn waste into treasure to protect the ecological environment.

(4) For the method provided by the present disclosure, it is easy to directionally adjust the intensity of the magnetic field by adjusting the current in the current-carrying coil, the number of coils, the coil resistance, and an applied voltage, and the method is applicable to all scenes of methane production in anaerobic digestion of organic wastes.

The concept, specific structure and achieved technical effects of the present disclosure are further described below with reference to the accompanying drawings, so as to fully understand the objective, features and effects of the present disclosure.

### BRIEF DESCRIPTION OF THE DRAWINGS

FIG. 1 is a structural schematic diagram of a device for improving methane production by anaerobic digestion through a magnetic field of a current-carrying coil according to a preferred embodiment of the present disclosure.

In the drawings: 1 feed pool; 2 feed pump; 3 connecting pipeline; 4 feed valve; 5 inoculum reservoir; 6 current-carrying coil; 7 stirring paddle; 8 stirring motor; 9 digester; 10 magnetic field measurement electrode; 11 magnetic field measurement instrument host; 12 constant-current power supply; 13 current output line; 14 current input line; 15 magnetic field; 16 gas flowmeter; 17 gas bag.

### DETAILED DESCRIPTION OF THE EMBODIMENTS

Multiple preferred embodiments of the present disclosure are introduced below with reference to the accompanying drawings of the specification, thus making technical contents thereof clearer and easier to understand. The present disclosure can be embodied in many different forms of embodiments, and the scope of protection of the present disclosure is not limited to the embodiments set forth herein.

In the accompanying drawings, structurally identical components are designated by the same reference numerals, and structurally or functionally similar components throughout are designated by similar reference numerals. The dimension and thickness of each component shown in the accompanying drawings are arbitrarily shown. The size and thickness of each component are not limited in the present disclosure. For the sake of clarity, the thickness of the components is exaggerated somewhat in some places in the accompanying drawings.

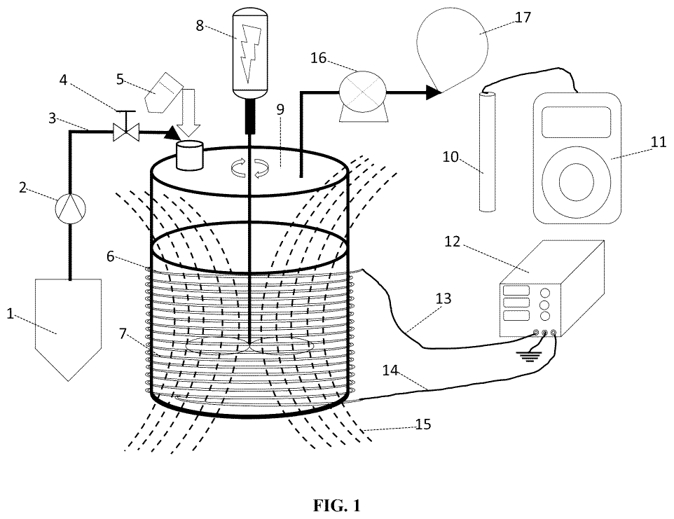

A structural schematic diagram of a device for improving methane production by anaerobic digestion through a magnetic field of a current-carrying coil is as shown in FIG. 1, including a feed pool 1, a feed pump 2, a connecting pipeline 3, a feed valve 4, an inoculum reservoir 5, a current-carrying coil 6, stirring paddles 7, a stirring motor 8, a digester 9, a magnetic field measurement electrode 10, a magnetic field measurement instrument host 11, a constant-current power supply 12, a current output line 13, a current input line 14, a magnetic field 15, a gas flowmeter 16, and a gas bag 17. A feed system is consisted of the feed pool 1, the feed pump 2, the connecting pipeline 3, the feed valve 4, and the inoculum reservoir 5. A stirring system is consisted of the stirring paddles 7 and the stirring motor 8, and the stirring paddles 7 are immersed in digestion feedstock to stir the digestion feedstock. A magnetic field generation system for generating a magnetic field 15 is consisted of the constant-current power supply 12, the current output line 13, the current input line 14, and the current-carrying coil 6. The current-carrying coil 6 is evenly wound around the digester 9 to generate the magnetic field 15, thus faster electron transfer for methane anabolic reaction is provided. The intensity of the magnetic field can be adjusted by adjusting a current in the current-carrying coil 6, so the best intensity of the magnetic field can be optimized and selected to efficiently and stably promote the methane anabolic process. The magnetic field measurement electrode 10 and the magnetic field measurement instrument host 11 are configured for measuring the magnetic field and feedback the measured magnetic field to the magnetic field generation system. The gas flowmeter 16 and the gas bag 17 are configured for evaluating methane yield in anaerobic digestion.

The specific use method of the device is specifically described below through Embodiments 1 to 4.

### Embodiment 1

Enhancement of Methane Production in Anaerobic Digestion of Kitchen Waste with Magnetic Field of Current-Carrying Coil

Collection and pretreatment of digestion feedstock: 18 kg of kitchen waste mixture is collected from the restaurant, and refractory components, such as plastics, metals, and shells, contained in the kitchen waste mixture are manually removed, the kitchen waste mixture is stirred with a high-speed stirrer to make a slurry mixture, and then the slurry mixture is stored in a refrigerator at 4° C. for later use.

Collection and pretreatment of anaerobic digestion inoculum: 20 L of digestion solution is taken from a large anaerobic digester for centralized treatment of kitchen waste as inoculum for this experiment, which is dispensed into inoculum reservoirs 5. Prior to the formal experiment, the inoculum is starved for 7 days in advance to release undecomposed organic matters, and an obtained inoculum is used as the anaerobic digestion inoculum.

Formation of anaerobic digestion system assisted by current-carrying coil: a glass bottle with a total volume of 1 L is used as a digester 9 (working volume of 0.8 L), and an enameled wire (current-carrying coil 6) with a length of 2 m is evenly wound around the glass bottle, and the outer coating of the two ends of the enameled wire with a length of 1 cm are scraped off with a blade to expose metal wire ends to be conveniently connected with an external constant-current power supply 12.

Start of anaerobic digestion experiment: an anaerobic digestion experiment is performed in an incubator at 37° C. The anaerobic digester assisted by the current-carrying coil is served as an experimental group, and an anaerobic digester without the assistance of the current-carrying coil is served as a control group, the obtained anaerobic digestion inoculum is inoculated into the digester 9, and the slurry kitchen waste feedstock is injected into the digester 9 through a feed pump 2. During the digestion process, the constant-current power supply 12 is turned on once every day, application time of the magnetic field of the current-carrying coil is set to 15 min each time, and a rated output voltage is 30 V. The intensity of the magnetic field is adjusted by adjusting a current in the current-carrying coil, and the current in the coil is proportional to the intensity of the magnetic field of the current-carrying coil. An actual output voltage is 5.39 V, an actual output current is 5.16 A, and an actual output power is 27.8 W. As can be known from the measurement of a magnetic field measurement electrode 10 and a magnetic field measurement instrument host 11, the intensity of the magnetic field 15 generated by the current-carrying coil 6 around the digester 9 is in the range of 0.3-70.4 mT. The intensity of the magnetic field 15 here is the sum of the intensity of the magnetic field at all points, and the closer the point is to the coil, the greater the intensity of the magnetic field; the farther the point is away from the coil, the smaller the intensity of the magnetic field. In this experiment, the actual intensity of the magnetic field acquired through the magnetic field measurement system is in the range of 0.3-70.4 mT. During culture, 40 mL of sample is taken every other day, then 5-10 g of slurry kitchen waste feedstock is added, and a certain volume of constant temperature water at 37° C. is added to make the working volume of the digester reach 0.8 L.

Results of anaerobic digestion experiment: the feedstock is digested and cultured for 35 days under the conditions that the digestion temperature is 37° C. and the stirring speed is 500 rpm, an average methane yield produced from anaerobic digestion without the current-carrying coil is 0.32 L CH₄/g-VS (VS refers to volatile solids), an average methane concentration in biogas is 53.2%, and an average carbon dioxide concentration in the biogas is 33.6%. Compared with the digestion with the same parameters of the digestion process without the assistance of the magnetic field of the current-carrying coil, the average methane yield of the anaerobic digestion with the assistance of the magnetic field of the current-carrying coil is increased by 30.8% (0.42 L CH₄/g-VS), the average methane concentration in the biogas is increased by 18.5%, and the average carbon dioxide concentration in the biogas is reduced by 14.7%.

The above steps are a complete period of anaerobic digestion of organic wastes. After a round of anaerobic digestion culture is finished, the digester can be cleaned, and then a new round of anaerobic digestion operation can be started to achieve the operation of the next fermentation period.

### Embodiment 2

Enhancement of Methane Production in Anaerobic Digestion of Agricultural Straw with Magnetic Field of Current-Carrying Coil

Collection and pretreatment of digestion feedstock: rice straw is collected from farmland as an anaerobic digestion feedstock, and the rice straw is placed in an oven at 60° C. for drying for 24-36 h, where the optimal drying time is 30 h. Afterwards, the dried rice straw is crushed with a high-speed crusher, and rice straw particles with the optimal particle diameter ranging from 0.4-1.2 mm are selected out by a sieve as biomass feedstock for subsequent anaerobic digestion, and the biomass feedstock is stored in a refrigerator at 4° C. for later use.

Collection and pretreatment of anaerobic digestion inoculum: a pre-domesticated digestion solution is taken from a 5 L anaerobic digester for treating the rice straw, the pre-domesticated digestion solution is served as the anaerobic digestion inoculum for this experiment, which is dispensed into inoculum reservoirs 5. Prior to the formal experiment, the inoculum is starved for 7 days in advance to release undecomposed organic matters, and an obtained inoculum is used as the anaerobic digestion inoculum.

Formation of anaerobic digestion system assisted by current-carrying coil: a glass bottle with a total volume of 1 L is used as a digester 9 (working volume of 0.8 L), and an enameled wire (current-carrying coil 6) with a length of 2 m is evenly wound around the glass bottle, and the outer coating of the two ends of the enameled wire with a length of 1 cm are scraped off with a blade to expose metal wire ends to be conveniently connected with an external constant-current power supply 12.

Start of anaerobic digestion experiment: an anaerobic digestion experiment is performed in an incubator at 37° C. The anaerobic digester assisted by the current-carrying coil is served as an experimental group, and an anaerobic digester without the assistance of the current-carrying coil is served as a control group, the obtained anaerobic digestion inoculum is inoculated into the digester 9, and the selected rice straw particle feedstock is injected into the digester 9 through a feed pump 2. During the digestion process, a switch of the constant-current power supply 12 is turned on once every day, application time of the magnetic field of the current-carrying coil is set to 10 min each time, and a rated output voltage is 30 V. The intensity of the magnetic field is adjusted by adjusting a current in the current-carrying coil, and the current in the coil is proportional to the intensity of the magnetic field of the current-carrying coil. An actual output voltage is 30 V, an actual output current is 1 A, and an actual output power is 30 W. As can be known from the measurement of a magnetic field measurement electrode 10 and a magnetic field measurement instrument host 11, the intensity of the magnetic field 15 generated by the current-carrying coil 6 around the digester 9 is in the range of 2.5-81.6 mT. The intensity of the magnetic field 15 here is the sum of the intensity of the magnetic field at all points, and the closer the point is to the coil, the greater the intensity of the magnetic field; the farther the point is away from the coil, the smaller the intensity of the magnetic field. In this experiment, the actual intensity of the magnetic field acquired through the magnetic field measurement system is in the range of 2.5-81.6 mT. During culture, 40 mL of sample is taken every other day, then 6-8 g of rice straw particle feedstock is added, and a certain volume of constant temperature water at 37° C. is added to make the working volume of the digester reach 0.8 L.

Results of anaerobic digestion experiment: the feedstock is digested and cultured for 45 days under the conditions that the digestion temperature is 37° C. and the stirring speed is 500 rpm, an average methane yield produced from anaerobic digestion without the current-carrying coil is 0.21 L CH₄/g-VS, an average methane concentration in biogas is 48.1%, and an average carbon dioxide concentration in the biogas is 33.6%. Compared with the digestion with the same parameters of the digestion process without the assistance of the magnetic field of the current-carrying coil, the average methane yield of the anaerobic digestion with the assistance of the magnetic field of the current-carrying coil is increased by 29.5% (0.27 L CH₄/g-VS), the average methane concentration in the biogas is increased by 22.4%, and the average carbon dioxide concentration in the biogas is reduced by 20.1%.

The above steps are a complete period of anaerobic digestion of organic wastes. After a round of anaerobic digestion culture is finished, the digester can be cleaned, and then a new round of anaerobic digestion operation can be started to achieve the operation of the next fermentation period.

### Embodiment 3

Enhancement of Methane Production in Anaerobic Digestion of Livestock Manure with Magnetic Field of Current-Carrying Coil

Collection and pretreatment of digestion feedstock: chicken manure is obtained from a chicken farm as an anaerobic digestion feedstock. The chicken manure is placed in an oven at 55° C. for drying for 24-48 h, where the optimal drying time is 36 h. Afterwards, the dried chicken manure is crushed with a high-speed crusher to obtain dried powder chicken manure as biomass feedstock for subsequent anaerobic digestion, and the biomass feedstock is stored in a refrigerator at 4° C. for later use.

Collection and pretreatment of anaerobic digestion inoculum: a pre-domesticated digestion solution is taken from a 5 L anaerobic digester for treating the livestock manure, the pre-domesticated digestion solution is served as the anaerobic digestion inoculum for this experiment, which is dispensed into inoculum reservoirs 5. Prior to the formal experiment, the inoculum is starved for 7 days in advance to release undecomposed organic matters, and an obtained inoculum is used as the anaerobic digestion inoculum.

Formation of anaerobic digestion system assisted by current-carrying coil: a glass bottle with a total volume of 1 L is used as a digester 9 (working volume of 0.8 L), and an enameled wire (current-carrying coil 6) with a length of 2 m is evenly wound around the glass bottle, and the outer coating of the two ends of the enameled wire with a length of 1 cm are scraped off with a blade to expose metal wire ends to be conveniently connected with an external constant-current power supply 12.

Start of anaerobic digestion experiment: an anaerobic digestion experiment is performed in an incubator at 37° C. The anaerobic digester assisted by the current-carrying coil is served as an experimental group, and an anaerobic digester without the assistance of the current-carrying coil is served as a control group, the obtained anaerobic digestion inoculum is inoculated into the digester 9, and the dried powder chicken manure feedstock is injected into the digester 9 through a feed pump 2. During the digestion process, a switch of the constant-current power supply 12 is turned on once every day, the preferably treatment time of the magnetic field is 5 min, and a rated output voltage is 30 V. The intensity of the magnetic field is adjusted by adjusting a current in the current-carrying coil, and the current in the coil is proportional to the intensity of the magnetic field of the current-carrying coil. An actual output voltage is 5.39 V, an actual output current is 5.16 A, and an actual output power is 27.8 W. As can be known from the measurement of a magnetic field measurement electrode 10 and a magnetic field measurement instrument host 11, the intensity of the magnetic field 15 generated by the current-carrying coil 6 around the digester 9 is in the range of 1.5-78.2 mT. The intensity of the magnetic field 15 here is the sum of the intensity of the magnetic field at all points, and the closer the point is to the coil, the greater the intensity of the magnetic field; the farther the point is away from the coil, the smaller the intensity of the magnetic field. In this experiment, the actual intensity of the magnetic field acquired through the magnetic field measurement system is in the range of 1.5-78.2 mT. During culture, 40 mL of sample is taken every other day, then 5 g of dried powder chicken manure feedstock is added, and a certain volume of constant temperature water at 37° C. is added to make the working volume of the digester reach 0.8 L. For the digester in the control group, except the constant-current power supply is not used, the other discharging and feeding operations are consistent with those in the experimental group.

Results of anaerobic digestion experiment: the feedstock is digested and cultured for 58 days under the conditions that the digestion temperature is 37° C. and the stirring speed is 500 rpm, an average methane yield produced from anaerobic digestion without the current-carrying coil is 0.20 L CH₄/g-VS, an average methane concentration in biogas is 43.7%, and an average carbon dioxide concentration in the biogas is 33.7%. Compared with the digestion with the same parameters of the digestion process without the assistance of the magnetic field of the current-carrying coil, the average methane yield of the anaerobic digestion with the assistance of the magnetic field of the current-carrying coil is increased by 35.0% (0.27 L CH₄/g-VS), the average methane concentration in the biogas is increased by 17.9%, and the average carbon dioxide concentration in the biogas is reduced by 20.6%.

The above steps are a complete period of anaerobic digestion of organic wastes. After a round of anaerobic digestion culture is finished, the digester can be cleaned, and then a new round of anaerobic digestion operation can be started to achieve the operation of the next fermentation period.

### Embodiment 4

Enhancement of Methane Production in Anaerobic Digestion of Livestock Manure with Magnetic Field of Current-Carrying Coil

Collection and pretreatment of digestion feedstock: chicken manure is obtained from a chicken farm as an anaerobic digestion feedstock. The chicken manure is placed in an oven at 55° C. for drying for 24-48 h, where the optimal drying time is 36 h. Afterwards, the dried chicken manure is crushed with a high-speed crusher to obtain dried powder chicken manure as biomass feedstock for subsequent anaerobic digestion, and the biomass feedstock is stored in a refrigerator at 4° C. for later use.

Collection and pretreatment of anaerobic digestion inoculum: a pre-domesticated digestion solution is taken from a 5 L anaerobic digester for treating the livestock manure, the pre-domesticated digestion solution is served as the anaerobic digestion inoculum for this experiment, which is dispensed into inoculum reservoirs 5. Prior to the formal experiment, the inoculum is starved for 7 days in advance to release undecomposed organic matters, and an obtained inoculum is used as the anaerobic digestion inoculum.

Formation of anaerobic digestion system assisted by current-carrying coil: a glass bottle with a total volume of 0.5 L is used as a digester 9 (working volume of 0.4 L), and an enameled wire (current-carrying coil 6) with a length of 1 m is evenly wound around the glass bottle, and the outer coating of the two ends of the enameled wire with a length of 1 cm are scraped off with a blade to expose metal wire ends to be conveniently connected with an external constant-current power supply 12.

Start of anaerobic digestion experiment: an anaerobic digestion experiment is performed in an incubator at 37° C. The anaerobic digester assisted by the current-carrying coil is served as an experimental group, and an anaerobic digester without the assistance of the current-carrying coil is served as a control group, the obtained anaerobic digestion inoculum is inoculated into the digester 9, and the dried powder chicken manure feedstock is injected into the digester 9 through a feed pump 2. During the digestion process, a switch of the constant-current power supply 12 is turned on once every day, the preferable treatment time of the magnetic field is 10 min, and a rated output voltage is 30 V. The intensity of the magnetic field is adjusted by adjusting a current in the current-carrying coil, and the current in the coil is proportional to the intensity of the magnetic field of the current-carrying coil. An actual output voltage is 1 V, an actual output current is 5.5 A, and an actual output power is 5.5 W. As can be known from the measurement of a magnetic field measurement electrode 10 and a magnetic field measurement instrument host 11, the intensity of the magnetic field 15 generated by the current-carrying coil 6 around the digester 9 is in the range of 0.3-28.2 mT. The intensity of the magnetic field 15 here is the sum of the intensity of the magnetic field at all points, and the closer the point is to the coil, the greater the intensity of the magnetic field; the farther the point is away from the coil, the smaller the intensity of the magnetic field. In this experiment, the actual intensity of the magnetic field acquired through the magnetic field measurement system is in the range of 0.3-28.2 mT. During culture, 20 mL of sample is taken every other day, then 2.5 g of dried powder chicken manure feedstock is added, and a certain volume of constant temperature water at 37° C. is added to make the working volume of the digester reach 0.4 L. For the digester in the control group, except the constant-current power supply is not used, the other discharging and feeding operations are consistent with those in the experimental group.

The above steps are a complete period of anaerobic digestion of organic wastes. After a round of anaerobic digestion culture is finished, the digester can be cleaned, and then a new round of anaerobic digestion operation can be started to achieve the operation of the next fermentation period. By repeating the above steps continuously, the sustainable operation of methane production from efficient anaerobic digestion of organic wastes assisted by current-carrying coil can be achieved.

The method has the advantages of simple operation, uniform magnetic field distribution, easy realization of industrial application and large-scale amplification, high methane production efficiency, good technical economy, etc. The intensity of the magnetic field can be regulated by dynamically adjusting the current in the current-carrying coil. The method is also compatible with the continuous operation of the whole anaerobic digestion process, and thus has an extremely high industrial application prospect.

The preferred specific embodiments of the present disclosure are described in detail above. It should be understood that those of ordinary skill in the art can make various modifications and changes according to the concept of the present disclosure without creative efforts. Therefore, any technical solution that can be obtained by those skilled in the art through logical analysis, reasoning or limited experiments on the basis of the prior art according to the concept of the present disclosure should be within the scope of protection determined by the claims.

What is claimed is:

1. A device for improving methane production in anaerobic digestion through a magnetic field of a current-carrying coil, comprising a feed system, a digester, a stirring system, a magnetic field generation system, a magnetic field measurement system, and a gas flow measurement system, wherein the feed system is connected to the digester, the stirring system comprises stirring paddles and a stirring motor, and the stirring paddles are immersed in digestion feedstock in the digester; the magnetic field generation system is evenly wound around the digester; the magnetic field measurement system is located outside the digester; and the gas flow measurement system is connected to an upper portion of the digester.

2. The device for improving methane production in anaerobic digestion through the magnetic field of the current-carrying coil according to claim 1, wherein the feed system comprises a feed pool, a feed pump, a connecting pipeline, a feed valve and an inoculum reservoir; the digestion feedstock is placed in the feed pool, the digestion feedstock is injected into the digester through the feed pump, and anaerobic digestion inoculum is added into the digester from the inoculum reservoir.

3. The device for improving methane production in anaerobic digestion through the magnetic field of the current-carrying coil according to claim 1, wherein the magnetic field generation system comprises a constant-current power supply, a power output line, the current-carrying coil, and a power input line; the current-carrying coil is evenly wound around the digester to excite the magnetic field to act on digestion microbial communities inside the digester.

4. The device for improving methane production in anaerobic digestion through the magnetic field of the current-carrying coil according to claim 1, wherein the magnetic field measurement system comprises a magnetic field measurement electrode, and a magnetic field measurement instrument host; the magnetic field measurement electrode is placed in the magnetic field of the digester, and configured for measuring actual intensity of the magnetic field and feedback data to the magnetic generation system to control intensity of the magnetic field in the digester; and the gas flow measurement system comprises a gas flowmeter, and a gas bag, and is configured for evaluating methane yield by anaerobic digestion.

5. A method for improving methane production in anaerobic digestion through a magnetic field of a current-carrying coil, wherein the device according to claim 1 is used in the method, and the method comprises the following steps:
Step 1, collecting and pretreating the digestion feedstock;
Step 2, collecting and pretreating anaerobic digestion inoculum;
Step 3, evenly winding the current-carrying coil around the digester;
Step 4, adding the digestion feedstock obtained in Step 1 and the anaerobic digestion inoculum obtained in Step 2 into the digester, turning on the magnetic field generation system and the stirring system, and measuring intensity of the magnetic field by the magnetic field measurement system, adjusting a current in the current-carrying coil to adjust the intensity of the magnetic field generated by the magnetic field generation system to an appropriate level, and starting the anaerobic digestion;
Step 5, during digestion, taking samples to measure digestion efficiency; and
Step 6, after the anaerobic digestion is finished, collecting gas in a gas bag, and measuring methane concentration and carbon dioxide concentration to evaluate average methane yield;
cleaning the digester after above steps are finished, and repeating Steps 1 to 6 for the next fermentation period.

6. The method according to claim 5, wherein the digestion feedstock in Step 1 is one of kitchen waste mixture, agricultural organic waste, or livestock manure, and a method for pretreating in Step 1 comprises one or more steps of screening, drying, high-speed stirring, or high-speed crushing the digestion feedstock.

7. The method according to claim 5, wherein Step 2 specifically comprises the following steps:
Step 2.1, taking out digestion solution from a digester of the same digestion feedstock domesticated in advance as the anaerobic digestion inoculum, and dispensing the anaerobic digestion inoculum into an inoculum reservoir; and
Step 2.2, prior to a formal experiment, starving the anaerobic digestion inoculum for 7 days in advance to release undecomposed organic matters;
wherein an obtained bacterial solution is used as the anaerobic digestion inoculum.

8. The method according to claim 5, wherein frequency of the magnetic field generation system applied in Step 4 is once a day in an entire digestion process, treatment time of the magnetic field generation system is 5-15 min each time, an output current of a constant-current power supply of the magnetic field generation system is 1-5.5 A, and an output voltage is 1-30 V; the intensity of the magnetic field is adjusted to 0.3-81.6 mT, digestion temperature is set to 37° C., and rotating speed of the stirring system is set to 500-550 rpm.

9. The method according to claim 5, wherein a method for taking samples in Step 5 is to take samples every other day, and after taking samples is finished, quantitative feedstock and constant-temperature water at 37° C. are added to ensure that working volume of the digester is the same as the initial one.

10. The method according to claim 5, wherein the average methane yield is increased by 29.5-35% compared with that without assistance of the magnetic field of the current-carrying coil.

11. The method according to claim 5, wherein the feed system comprises a feed pool, a feed pump, a connecting pipeline, a feed valve and an inoculum reservoir; the digestion feedstock is placed in the feed pool, the digestion feedstock is injected into the digester through the feed pump, and the anaerobic digestion inoculum is added into the digester from the inoculum reservoir.

12. The method according to claim 5, wherein the magnetic field generation system comprises a constant-current power supply, a power output line, the current-carrying coil, and a power input line; the current-carrying coil is evenly wound around the digester to excite the magnetic field to act on digestion microbial communities inside the digester.

13. The method according to claim 5, wherein the magnetic field measurement system comprises a magnetic field measurement electrode, and a magnetic field measurement instrument host; the magnetic field measurement electrode is placed in the magnetic field of the digester, and configured for measuring actual intensity of the magnetic field and feedback data to the magnetic generation system to control the intensity of the magnetic field in the digester; and the gas flow measurement system comprises a gas flowmeter, and the gas bag, and is configured for evaluating methane yield by anaerobic digestion.

14. The method according to claim 6, wherein the feed system comprises a feed pool, a feed pump, a connecting pipeline, a feed valve and an inoculum reservoir; the digestion feedstock is placed in the feed pool, the digestion feedstock is injected into the digester through the feed pump, and the anaerobic digestion inoculum is added into the digester from the inoculum reservoir.

15. The method according to claim 6, wherein the magnetic field generation system comprises a constant-current power supply, a power output line, the current-carrying coil, and a power input line; the current-carrying coil is evenly wound around the digester to excite the magnetic field to act on digestion microbial communities inside the digester.

16. The method according to claim 6, wherein the magnetic field measurement system comprises a magnetic field measurement electrode, and a magnetic field measurement instrument host; the magnetic field measurement electrode is placed in the magnetic field of the digester, and configured for measuring actual intensity of the magnetic field and feedback data to the magnetic generation system to control the intensity of the magnetic field in the digester; and the gas flow measurement system comprises a gas flowmeter, and the gas bag, and is configured for evaluating methane yield by anaerobic digestion.

17. The method according to claim 7, wherein the feed system comprises a feed pool, a feed pump, a connecting pipeline, a feed valve and the inoculum reservoir; the digestion feedstock is placed in the feed pool, the digestion feedstock is injected into the digester through the feed pump, and the anaerobic digestion inoculum is added into the digester from the inoculum reservoir.

18. The method according to claim 7, wherein the magnetic field generation system comprises a constant-current power supply, a power output line, the current-carrying coil, and a power input line; the current-carrying coil is evenly wound around the digester to excite the magnetic field to act on digestion microbial communities inside the digester.

19. The method according to claim 7, wherein the magnetic field measurement system comprises a magnetic field measurement electrode, and a magnetic field measurement instrument host; the magnetic field measurement electrode is placed in the magnetic field of the digester, and configured for measuring actual intensity of the magnetic field and feedback data to the magnetic generation system to control the intensity of the magnetic field in the digester; and
the gas flow measurement system comprises a gas flowmeter, and the gas bag, and is configured for evaluating methane yield by anaerobic digestion.

20. The method according to claim 8, wherein the feed system comprises a feed pool, a feed pump, a connecting pipeline, a feed valve and the inoculum reservoir; the digestion feedstock is placed in the feed pool, the digestion feedstock is injected into the digester through the feed pump, and the anaerobic digestion inoculum is added into the digester from the inoculum reservoir.
